(19) United States
(12) Patent Application Publication
(10) Pub. No.: US 2016/0175532 A1
(43) Pub. Date: Jun. 23, 2016

(54) Non Slip Syringe Assembly

(71) Applicant: Michelle Gutierrez, Cerritos, CA (US)

(72) Inventor: Michelle Gutierrez, Cerritos, CA (US)

(21) Appl. No.: 14/573,592

(22) Filed: Dec. 17, 2014

(51) Int. Cl. A61M 5/31

(52) U.S. Cl. CPC A61M 5/3137

(57) ABSTRACT

A non-slip syringe assembly for providing greater control of the syringe includes a syringe structured to have an outer wall. A gripping layer is coupled to the outer wall. The gripping layer may be gripped. A needle is movably coupled to the syringe. The needle is positionable between a deployed position and a stored position.

### BACKGROUND OF THE DISCLOSURE

### Field of the Disclosure

The disclosure relates to syringe devices and more particularly pertains to a new syringe device for providing greater control of the syringe.

### SUMMARY OF THE DISCLOSURE

An embodiment of the disclosure meets the needs presented above by generally comprising a syringe structured to have an outer wall. A gripping layer is coupled to the outer wall. The gripping layer may be gripped. A needle is movably coupled to the syringe. The needle is positionable between a deployed position and a stored position.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF THE DRAWINGS

The disclosure will be better understood and objects other than those set forth above will become apparent when consideration is given to the following detailed description thereof. Such description makes reference to the annexed drawings wherein:

FIG. 1 is a perspective view of a non-slip syringe assembly according to an embodiment of the disclosure.

FIG. 2 is a bottom view of an embodiment of the disclosure.

FIG. 3 is a front view of an embodiment of the disclosure.

FIG. 4 is a left side view of an embodiment of the disclosure.

FIG. 5 is a cross sectional view taken along line 5-5 of FIG. 2 of an embodiment of the disclosure.

FIG. 6 is a cross-sectional view similar to FIG. 5 of an embodiment of the disclosure.

### DESCRIPTION OF THE PREFERRED EMBODIMENT

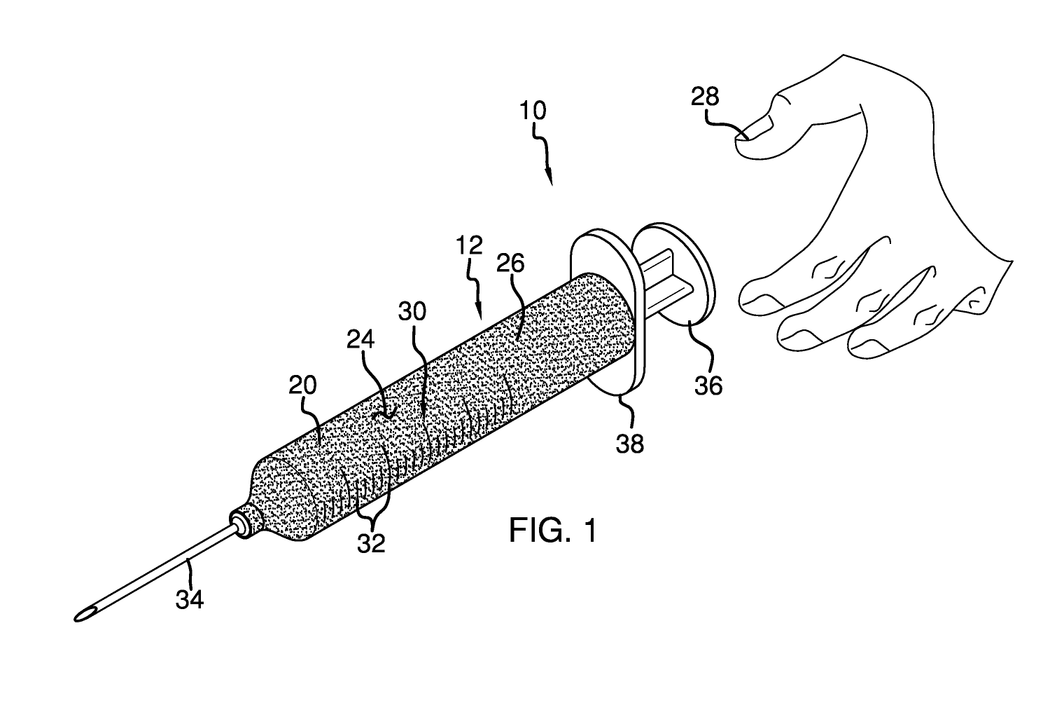

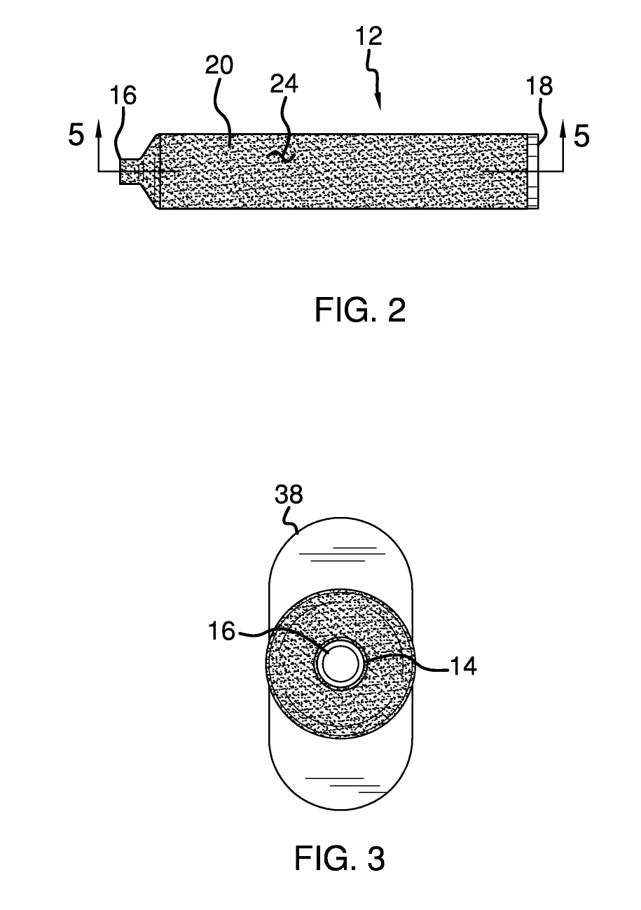

With reference now to the drawings, and in particular to FIGS. 1 through 6 thereof, a new syringe device embodying the principles and concepts of an embodiment of the disclosure and generally designated by the reference numeral 10 will be described.

As best illustrated in FIGS. 1 through 6, the non-slip syringe assembly 10 generally comprises a syringe 12 that has an outer wall 14 extending between each of a first end 16 and a second end 18 of the syringe 12. Each of the first 16 and second ends 18 of the syringe 12 is open. The syringe 12 is substantially hollow. Additionally, the outer wall 14 of the syringe 12 tapers toward the second end 18 of the syringe 12. The first end 16 of the syringe 12 has a diameter that is less than a diameter of the second end 18 of the syringe 12.

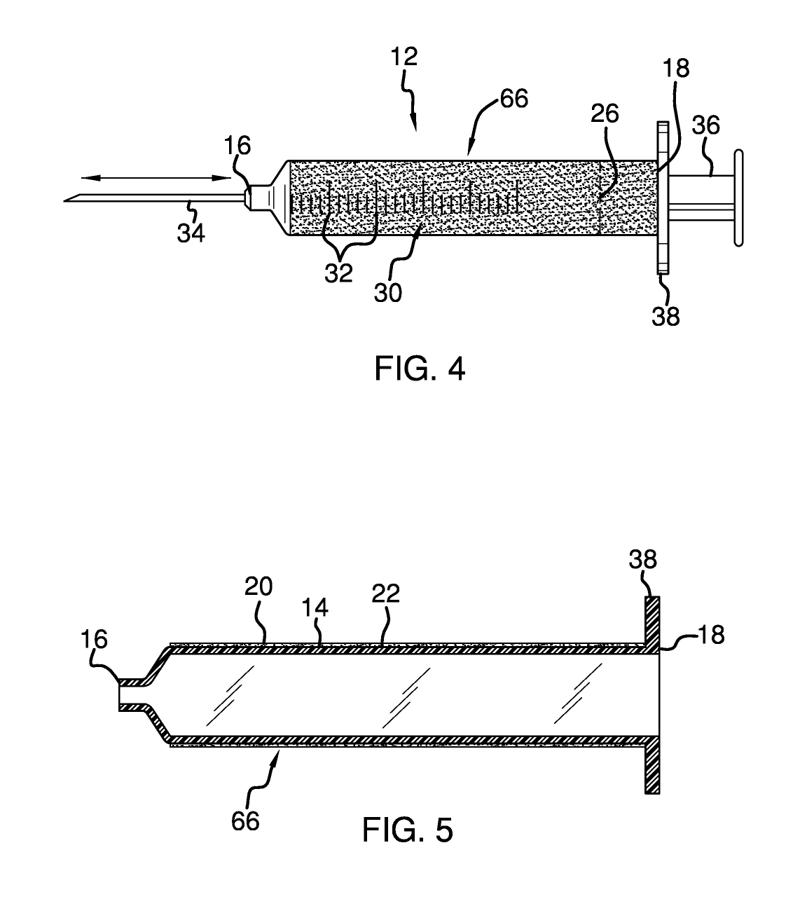

As shown in FIGS. 1 through 5, a gripping layer 20 is provided. The gripping layer 20 is coupled to an outer surface 22 of the outer wall 14 of the syringe 12. The gripping layer 20 may be gripped. As shown in FIGS. 4 and 5, the gripping layer 20 extends along a barrel section 66 of the outer wall 14 between the first 16 and second 18 ends of the syringe 12. Alternatively, as shown in FIGS. 1 through 3, the gripping layer 20 may be coextensive with the outer wall 14 covering an entirety of the outer wall 14 of the syringe 12.

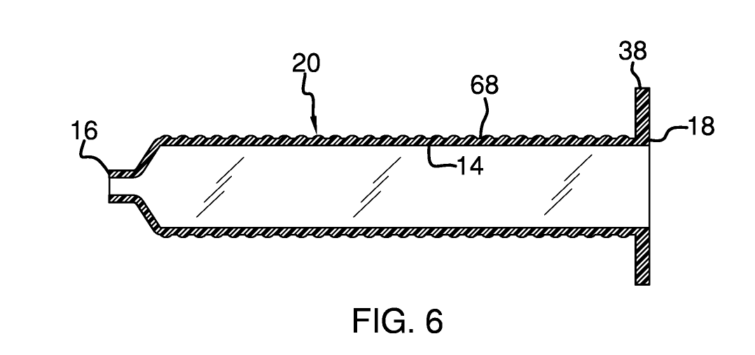

An exposed surface 24 of the gripping layer 20 comprises a rough texture. Additionally, the gripping layer 20 may be comprised of a translucent material. The gripping layer 20 allows a material 26 contained within the syringe 12 to be visible to a user 28. The material 26 contained within the syringe 12 may be a liquid medication of any conventional design. As shown in FIG. 6, the gripping layer 20 may be integrally formed with the outer wall 14 by indentations, or the like, providing roughness to an exterior surface 68 of the outer wall 14.

Indicia 30 are printed on the exposed surface 24 of the gripping layer 20. The

Indicia 30 comprise graduated volume marks 32. The graduated volume marks 32 extend between the first 16 and second 18 ends of the syringe 12. The graduated volume marks 32 indicate a volume of the material 26 contained within the syringe 12.

A needle 34 is movably coupled to the first end 16 of the syringe 12. The needle 34 is in fluid communication with an interior of the syringe 12. Additionally, the needle 34 may be a hypodermic needle of any conventional design. The needle 34 is positionable in a deployed position. The needle 34 extends outwardly from the first end 16 of the syringe 12. The needle 34 is positionable in a stored position. Moreover, the needle 34 is completely contained within the syringe 12 in the stored position.

A plunger 36 is provided. The plunger 36 extends into the second end 18 of the syringe 12. The plunger 36 is slidably positioned in the syringe 12. Additionally, the plunger 36 may urge the material 26 outwardly from the needle 34.

A grip 38 is provided. The grip 38 is coupled to and extends outwardly from the outer wall 14 of the syringe 12. The grip 38 is positioned adjacent to the second end 18 of the syringe 12. The grip 38 is gripped while the plunger 36 is being manipulated.

In use, the user 28 grips the gripping layer 20 while utilizing the syringe 12. The gripping layer 20 gives the user 28 greater control over the syringe 12 while the syringe 12 is utilized. The needle 34 is positioned in the deployed position to utilize the syringe 12. Additionally, the needle 34 is positioned in the stored position when the needle 34 is not being utilized. The needle 34 cannot accidentally pierce an individual when the needle 34 is in the stored position.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A non-slip syringe assembly comprising:
a syringe structured to have an outer wall, said outer wall of said syringe extending between each of a first end and a second end of said syringe, each of said first and second ends of said syringe being open, said syringe being substantially hollow;
a gripping layer coupled to said outer wall such that said gripping layer covers and extends around said outer wall providing a cylindrical outer surface, said gripping layer having a rough texture such that said gripping layer is configured to enhance gripping of said syringe, said gripping layer extending between said first and second ends of said syringe such that said gripping layer completely covers said outer wall of said syringe; and
a needle coupled to said syringe such that said needle extends from said syringe.

2. (canceled)

3. The assembly according to claim 1, further comprising said outer wall of said syringe tapering toward said second end of said syringe such that said first end of said syringe has a diameter being less than a diameter of said second end of said syringe.

4. (canceled)

5. The assembly according to claim 1, further comprising said needle extending outwardly from said first end of said syringe.

6. (canceled)

7. The assembly according to claim 1, further comprising a plunger extending into said second end of said syringe such that said plunger is slidably positioned in said syringe wherein said plunger is configured to urge a material outwardly from said needle.

8. A non-slip syringe assembly comprising:
a syringe having an outer wall extending between each of a first end and a second end of said syringe, each of said first and second ends of said syringe being open, said syringe being substantially hollow, said outer wall of said syringe tapering toward said second end of said syringe such that said first end of said syringe has a diameter being less than a diameter of said second end of said syringe;
a gripping layer coupled to said outer wall such that said gripping layer covers and extends around said outer wall providing a cylindrical outer surface such that said gripping layer is configured to be gripped, said gripping layer extending between said first and second ends of said syringe such that said gripping layer completely covers said outer wall of said syringe, said gripping layer having a rough texture;
a needle coupled to said first end of said syringe such that said needle is in fluid communication with an interior of said syringe; and
a plunger extending into said second end of said syringe such that said plunger is slidably positioned in said syringe wherein said plunger is configured to urge a material outwardly from said needle.
